(19) United States
(12) Patent Application Publication
(10) Pub. No.: US 2026/0320795 A1
(43) Pub. Date: Oct. 15, 2026

(54) INFORMATION PROCESSING SYSTEM, INFORMATION PROCESSING APPARATUS, INFORMATION PROCESSING METHOD, AND STORAGE MEDIUM

(71) Applicant: CANON KABUSHIKI KAISHA, Tokyo (JP)

(72) Inventors: YOSHIHIKO IWASE, Kanagawa (JP); HIROKI UCHIDA, Tokyo (JP)

(21) Appl. No.: 19/684,221

(22) Filed: May 21, 2026

Related U.S. Application Data
(63) Continuation of application No. PCT/JP2024/040900, filed on Nov. 19, 2024.

(30) Foreign Application Priority Data
Nov. 30, 2023 (JP) 2023-202721

(51) Int. Cl. G06T 7/00; G16H 30/20

(52) U.S. Cl. CPC G06T 7/0012; G16H 30/20; G06T 2207/10101; G06T 2207/30041

(57) ABSTRACT

An information processing apparatus includes a generation unit configured to generate, based on a tomographic image of an eye to be examined, a first analysis result having two-dimensional thickness data of a retinal layer of the eye to be examined, and generate a second analysis result different from the first analysis result using a machine learning model trained with a plurality of thickness maps corresponding to a plurality of normal eyes and the first analysis result, and a display control unit configured to control a display unit to display information regarding the second analysis result.

### CROSS-REFERENCE TO RELATED APPLICATIONS

This application is a Continuation of International Patent Application No. PCT/JP2024/040900, filed Nov. 19, 2024, which claims the benefit of Japanese Patent Application No. 2023-202721, filed Nov. 30, 2023, both of which are hereby incorporated by reference herein in their entirety.

### BACKGROUND

### FIELD OF THE TECHNOLOGY

The present disclosure relates to an information processing system, an information processing apparatus, an information processing method, and a storage medium.

### DESCRIPTION OF THE RELATED ART

As ophthalmic apparatuses, there have been put into practical use, an apparatus for acquiring a two-dimensional fundus image of an eye to be examined (hereinafter, referred to as a fundus camera apparatus) and apparatuses for acquiring a tomographic image of an eye to be examined using optical coherence tomography (OCT) with low coherence light (hereinafter, referred to as an OCT apparatus).

Measuring thicknesses of respective layers of a retina from tomographic images of the retina captured by these apparatuses enables quantitative diagnosis of a degree of progression of a disease such as glaucoma and a degree of recovery after treatment. In order to quantitatively measure thicknesses of these layers, thicknesses of retinal layers obtained from an eye to be examined are compared with stored statistical values of thicknesses of retinal layers in normal eyes (normal-eye database), and a portion deviating from the statistical values of thicknesses of retinal layers of normal eyes is displayed. When an axial length of an eyeball increases, the eyeball elongates to generally reduce retinal thicknesses, and a difference may become large as compared with the general statistical values of retinal thicknesses of normal eyes.

Japanese Patent Laid-Open No. 2015-84865 describes a technique in which, in addition to a database (first database) storing fundus layer thickness information regarding a plurality of eyes having long axial lengths, a normal-eye database (second database) is provided, and analysis information is obtained by referring to one of the databases.

The statistical values of thicknesses stored in the normal-eye database are average values for a plurality of eyes. On the other hand, normal values of thicknesses vary among individuals. For example, even when a difference from the statistical values of thicknesses stored in the normal-eye database is observed, the thicknesses may be normal for the individual. In such cases, when a diagnosis is made by referring to the normal-eye database, an eye to be examined that is normal may be diagnosed as abnormal.

In addition to thicknesses, a trajectory shape of nerve fibers also varies among individuals. For example, an arcuate trajectory shape of nerve fibers is considered normal, whereas, even when the trajectory shape is not arcuate, the trajectory shape may be normal for the individual. In such cases, for example, when a diagnosis is made by referring to the normal-eye database, an eye to be examined that is normal may be diagnosed as abnormal.

### SUMMARY

The present disclosure is directed to improving the accuracy of diagnosis.

An information processing apparatus includes a generation unit configured to generate, based on a tomographic image of an eye to be examined, a first analysis result having two-dimensional thickness data of a retinal layer of the eye to be examined, and generate a second analysis result different from the first analysis result using a machine learning model trained with a plurality of thickness maps corresponding to a plurality of normal eyes and the first analysis result, and a display control unit configured to control a display unit to display information regarding the second analysis result.

Features of the present disclosure will become apparent from the following description of embodiments with reference to the attached drawings.

### BRIEF DESCRIPTION OF THE DRAWINGS

FIG. 1 is a diagram illustrating an example of a schematic configuration of an image processing system (information processing system) according to a first embodiment.

FIG. 2A is a diagram illustrating a structure of an eye.

FIG. 2B is a diagram illustrating a tomographic image.

FIG. 2C is a diagram illustrating a fundus image.

FIG. 3 is a flowchart illustrating a flow of processing in the image processing system.

FIG. 4 is a diagram illustrating a screen for displaying an image according to the first embodiment.

FIG. 5A is a diagram illustrating a concept of an image generation model included in a generation unit in an image processing apparatus (information processing apparatus) according to the first embodiment.

FIG. 5B is a diagram illustrating a concept of an image generation model included in the generation unit in the image processing apparatus (information processing apparatus) according to the first embodiment.

FIG. 6 is a diagram illustrating training of the image generation model included in the generation unit in the image processing apparatus (information processing apparatus) according to the first embodiment.

FIG. 7A is a diagram illustrating inference of the image generation model included in the generation unit in the image processing apparatus (information processing apparatus) according to the first embodiment.

FIG. 7B is a diagram illustrating inference of the image generation model included in the generation unit in the image processing apparatus (information processing apparatus) according to the first embodiment.

FIG. 8 is a diagram illustrating an example of a schematic configuration of an ophthalmic apparatus according to a second embodiment.

FIG. 9 is a diagram illustrating a screen for displaying an image according to the second embodiment.

FIG. 10A is a diagram illustrating an image generated by a difference calculation unit in an image processing apparatus (information processing apparatus) according to the second embodiment.

FIG. 10B is a diagram illustrating an image generated by a difference calculation unit in the image processing apparatus (information processing apparatus) according to the second embodiment.

FIG. 11 is a diagram illustrating an example of a schematic configuration of an ophthalmic apparatus according to a third embodiment.

FIG. 12A is a diagram illustrating a concept of an image generation model included in a generation unit in an image processing apparatus (information processing apparatus) according to the third embodiment.

FIG. 12B is a diagram illustrating a concept of an image generation model included in the generation unit in the image processing apparatus (information processing apparatus) according to the third embodiment.

FIG. 13 is a diagram illustrating a screen for displaying an image in a fourth embodiment.

### DESCRIPTION OF THE EMBODIMENTS

Hereinafter, embodiments for implementing the present technique will be described in detail with reference to the drawings. However, dimensions, materials, shapes, relative positions of components, and the like described in the embodiments below are optional and can be changed depending on a configuration of an apparatus to which the present technique is applied or various conditions. In the drawings, the same reference numerals are used across the drawings to denote elements that are identical or functionally similar.

### First Embodiment

In the present embodiment, as an example of an ophthalmic apparatus according to the present technique, an ophthalmic apparatus that performs both optical coherence tomography (OCT) imaging and fundus imaging using visible light will be described. The ophthalmic apparatus of the present embodiment detects retinal layers from captured OCT data and generates two-dimensional thickness data of the retinal layers from information about the detected layers. Then, the ophthalmic apparatus generates two-dimensional thickness data inferred by an image generation model from the two-dimensional thickness data.

An image processing system (information processing system) in the present embodiment may be configured such that the ophthalmic apparatus (composite examination unit) according to the disclosed technology is connected to a network, receives an order (examination instruction) from a doctor's personal computer (PC), performs an examination of an eye to be examined in accordance with the order, and transmits a result of the examination to the doctor's PC.

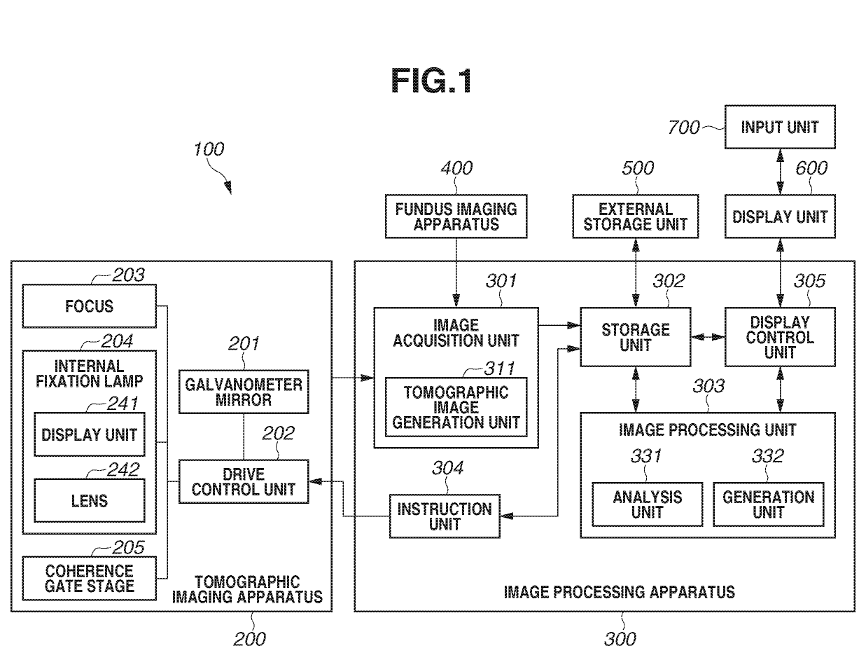

FIG. 1 is a diagram illustrating a configuration of an image processing system 100 (information processing system) including an image processing apparatus 300 according to the present embodiment. As illustrated in FIG. 1, the image processing system 100 is configured such that the image processing apparatus 300 is communicatively connected via an interface to a tomographic imaging apparatus (also referred to as an OCT apparatus) 200, a fundus imaging apparatus 400, an external storage unit 500, a display unit 600, and an input unit 700. Here, being “communicatively connected” includes not only direct communication between apparatuses but also communication via other devices. The image processing apparatus 300 is an example of an information processing apparatus.

The tomographic imaging apparatus 200 is an apparatus that captures a tomographic image of an eye. An apparatus used as the tomographic imaging apparatus 200 may be, for example, a spectral-domain OCT (SD-OCT) apparatus or a swept-source OCT (SS-OCT) apparatus. Since the tomographic imaging apparatus 200 is a known apparatus, a detailed description thereof will be omitted, and tomographic image capturing performed in accordance with an instruction from the image processing apparatus 300 will be described here.

In FIG. 1, a galvanometer mirror 201 is used to scan the fundus with measurement light and defines an OCT imaging range on the fundus. The drive control unit 202 defines an imaging range in a planar direction on the fundus and the number of scan lines (a scanning speed in the planar direction) by controlling a drive range and a speed of the galvanometer mirror 201. For the sake of simplicity, the galvanometer mirror is illustrated here as a single unit, but the galvanometer mirror actually includes two mirrors, i.e., an X scan mirror and a Y scan mirror, which can scan a desired area on the fundus with measurement light.

A focus 203 is used to focus on retinal layers of the fundus through an anterior segment of an eye serving as a subject. The measurement light is focused on the retinal layers of the fundus through the anterior segment of the eye, which is the subject, by a focus lens (not illustrated). The measurement light having irradiated the fundus returns after being reflected and scattered by the respective retinal layers.

An internal fixation lamp 204 includes a display unit 241 and a lens 242. As the display unit 241, a plurality of light-emitting diodes (LDs) arranged in a matrix is used. A lit position of the light-emitting diode(s) is changed in accordance with an area to be imaged under the control of the drive control unit 202. Light from the display unit 241 is guided to the eye to be examined via the lens 242. The light emitted from the display unit 241 has a wavelength of 520 nanometers (nm), and the drive control unit 202 causes a desired pattern to be displayed.

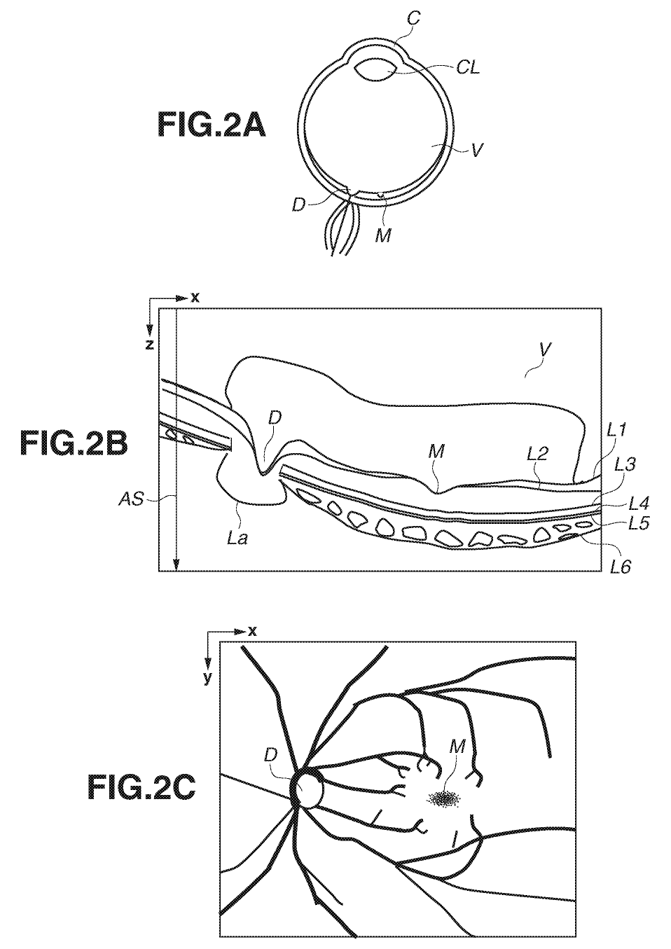

A coherence gate stage 205 is controlled by the drive control unit 202 to accommodate differences in an axial length of the eye to be examined, and the like. The coherence gate represents a position where measurement light and reference light are equal in optical path length in OCT. Furthermore, as an imaging method, controlling a position of the coherence gate controls whether imaging is performed on a retinal-layer side or on a deeper side than the retinal layers. Here, a structure of an eye and images acquired by the image processing system will be described with reference to FIGS. 2A-2C.

FIG. 2A is a schematic view of an eyeball. FIG. 2A illustrates a cornea C, a crystalline lens CL, a vitreous body V, a macular region M (a central portion of a macula represents a fovea), and an optic nerve head D. The tomographic imaging apparatus 200 according to the present embodiment will be described mainly regarding a case where a posterior pole portion of a retina including the vitreous body, the macular region, and the optic nerve head is imaged. Although not described in the present embodiment, the tomographic imaging apparatus 200 can also image an anterior segment of an eye including the cornea and crystalline lens.

FIG. 2B illustrates an example of a tomographic image acquired when the tomographic imaging apparatus 200 images a retina. FIG. 2B illustrates an A-scan AS that is a unit of image acquisition in an OCT tomographic image. A plurality of A-scans constitutes one B-scan. The B-scan is referred to as a tomographic image (or a tomographic view). FIG. 2B illustrates the vitreous body V, the macular region M, the optic nerve head D, and a lamina cribrosa La. In addition, FIG. 2B illustrates a boundary L1 between an internal limiting membrane (ILM) and a nerve fiber layer (NFL), a boundary L2 between the nerve fiber layer and a ganglion cell layer (GCL), a photoreceptor inner segment/outer segment (ISOS) junction L3, a retinal pigment epithelium layer (RPE) layer L4, a Bruch's membrane (BM) L5, and a choroid L6. In the tomographic image, the horizontal axis (a main scanning direction of OCT) is defined as an x-axis, and the vertical axis (a depth direction) is defined as a z-axis.

FIG. 2C illustrates an example of a fundus image acquired by the fundus imaging apparatus 400. The fundus imaging apparatus 400 is an apparatus that captures a fundus image of an eye, and examples of such apparatus include a fundus camera and a scanning laser ophthalmoscope (SLO). FIG. 2C illustrates the macular region M, the optic nerve head D, and retinal blood vessels presented by thick curved lines. In the fundus image, the horizontal axis (a main scanning direction of OCT) is defined as an x-axis, and the vertical axis (a secondary scanning direction of OCT) is defined as a y-axis. The tomographic imaging apparatus 200 and the fundus imaging apparatus 400 may be integrated with each other or may be separated from each other.

The image processing apparatus 300 includes an image acquisition unit 301, a storage unit 302, an image processing unit 303, an instruction unit 304, and a display control unit 305. The image acquisition unit 301 includes a tomographic image generation unit 311, acquires signal data of a tomographic image captured by the tomographic imaging apparatus 200, and generates a tomographic image by performing signal processing. The image acquisition unit 301 also acquires fundus image data captured by the fundus imaging apparatus 400. The image acquisition unit 301 then stores the generated tomographic image and the fundus image in the storage unit 302. The storage unit 302 stores various types of information and various types of data including image data. The storage unit 302 further stores programs for realizing various functions when being read and executed by a processing circuit, for example. For example, the storage unit 302 is implemented by a semiconductor memory element such as a random access memory (RAM) or a flash memory, or by a hard disk, an optical disk, or the like. The storage unit 302 stores statistical values of retinal layer thicknesses for a plurality of eyes (normal-eye database).

The image processing unit 303 includes an analysis unit 331 and a generation unit 332. The analysis unit 331 detects retinal layers from captured OCT data and generates two-dimensional thickness data of the retinal layers (hereinafter, also referred to as a thickness map) as a first analysis result using the information regarding the detected layers. The generation unit 332 has a data generation engine (a machine learning model) including a machine learning engine, and generates a thickness map as a second analysis result by using the thickness map generated as the first analysis result by the analysis unit 331.

The display control unit 305 controls the display unit 600. The display control unit 305 controls the display unit 600 to display information related to the second analysis result. The information related to the second analysis result is, for example, the thickness map itself as the second analysis result. Furthermore, the information related to the second analysis result is, for example, a comparison result obtained by comparing the second analysis result with the first analysis result. The comparison result here includes difference information between the second analysis result and the first analysis result, for example.

The analysis unit 331 and the generation unit 332 do not necessarily need to be separate components and may be implemented as a single generation unit. Alternatively, the generation unit 332 may be provided in an external apparatus different from the image processing apparatus 300. In this configuration, the image processing apparatus 300 outputs the first analysis result generated by the analysis unit 331 to the external apparatus. The external apparatus generates a second analysis result using the input first analysis result, and outputs the generated second analysis result to the image processing apparatus 300. The image processing apparatus 300 then displays the input second analysis result on the display unit 600.

The image processing apparatus 300 may be configured using a general-purpose computer, for example. Alternatively, the image processing apparatus 300 may be configured using a dedicated computer for an ophthalmic apparatus. The image processing apparatus 300 includes a storage medium including a processor (not illustrated) and a memory such as an optical disk or read only memory (ROM). The processor may be a central processing unit (CPU), a micro processing unit (MPU), or the like. The processor is not limited to a CPU or an MPU and may be, for example, a graphics processing unit (GPU) or a field-programmable gate array (FPGA). Each component of the image processing apparatus 300 other than the storage unit 302 may be configured by a software module executed by a processor such as a CPU, MPU, or GPU. Furthermore, each component may be configured by a circuit that performs a specific function, such as an application specific integrated circuit (ASIC), or by an independent apparatus, or the like. The processor or circuit may include a digital signal processor (DSP), a data flow processor (DFP), or a neural processing unit (NPU).

The external storage unit 500 stores information regarding an eye to be examined (information regarding a patient such as a name, race, age, sex, and measurement values regarding an eye such as an axial length), captured image data, imaging parameters, image analysis parameters, and parameters set by an operator in an associated manner.

The input unit 700 is, for example, a mouse, a keyboard, or a touch operation screen. An operator issues instructions to the image processing apparatus 300, the tomographic imaging apparatus 200, and the fundus imaging apparatus 400 via the input unit 700.

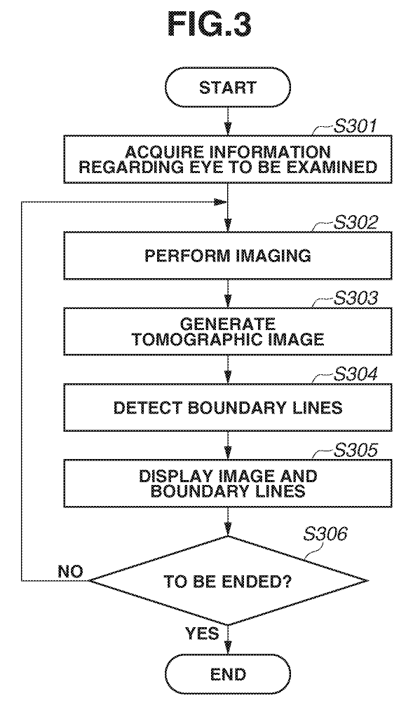

Next, a procedure of processing performed by the image processing apparatus 300 of the present embodiment will be described with reference to FIG. 3. FIG. 3 is a flowchart illustrating a flow of operation processing in the entire system, including image capturing and analysis, according to the present embodiment.

### Step S301

In step S301, an eye information acquisition unit (not illustrated) acquires, from an external source, a subject identification number as information for identifying an eye to be examined. Based on the subject identification number, the eye information acquisition unit acquires information regarding the eye to be examined stored in the external storage unit 500 and stores the acquired information in the storage unit 302.

### Step S302

In step S302, the eye to be examined is scanned and imaged. The scanning of the eye to be examined is performed such that, when an operator selects a scan start (not illustrated), the tomographic imaging apparatus 200 controls the drive control unit 202 to operate the galvanometer mirror 201 and scans the eye to be examined to capture a tomographic image. The galvanometer mirror 201 includes an X scanner for a horizontal direction and a Y scanner for a vertical direction. Thus, changing the respective orientations of these scanners makes it possible to perform scanning in each of the horizontal direction (X) and the vertical direction (Y) in an apparatus coordinate system. Furthermore, changing the orientations of these scanners simultaneously makes it possible to perform scanning in a direction obtained by combining the horizontal direction and the vertical direction, so that scanning can be performed in any direction on a fundus plane.

To perform imaging, various imaging parameters are adjusted. Specifically, at least a position of the internal fixation lamp, a scan range, a scan pattern, a coherence gate position, and a focus are set. The drive control unit 202 controls the position of the internal fixation lamp 204 by controlling the light-emitting diodes of the display unit 241 so that imaging is performed at a macular center or an optic nerve head. As the scan pattern, a scan pattern such as a raster scan, a radial scan, or a cross scan for capturing a three-dimensional volume is set. In the present embodiment, a case where imaging is performed using, as the scan pattern, a 3D scan for capturing a three-dimensional volume will be described. After the adjustment of these imaging parameters is completed, the operator selects an imaging start (not illustrated) to perform imaging. Although not described in the present embodiment, scanning of the eye to be examined is performed while tracking the eye to reduce an influence of fixational eye movements.

### Step S303

In step S303, a tomographic image is generated. The tomographic image generation unit 311 generates a tomographic image by performing a general reconstruction process on each interference signal.

First, the tomographic image generation unit 311 performs fixed pattern noise reduction on an interference signal. The fixed pattern noise reduction is performed by averaging a plurality of detected A-scan signals to extract fixed pattern noise and subtracting the extracted fixed pattern noise from an input interference signal. Next, the tomographic image generation unit 311 performs a desired window function process to optimize depth resolution and dynamic range, which have a trade-off relationship when a Fourier transform is performed over a finite interval. Next, the tomographic image generation unit 311 generates a tomographic signal by performing a fast Fourier transform (FFT) process.

### Step S304

In step S304, the analysis unit 331 performs layer detection. The analysis unit 331 detects boundary lines of retinal layers in a plurality of tomographic images captured by the tomographic imaging apparatus 200. The boundaries of the retinal layers are detected by using a machine learning model such as a deep learning model, or by image processing.

For example, when data is input to a machine learning model, data according to a design of the machine learning model is output. Output data that is highly likely to correspond to the input data is output according to a tendency trained from training data. In addition, a likelihood is output as a numerical value for each type of output data trained from the training data. Specifically, for example, when a tomographic image acquired by OCT is input to a machine learning model trained with first training data, an imaging site label of an imaging area captured in the image is output, or, depending on the design, a probability for each imaging site label is output. In addition, for example, when an OCT tomographic image is input to a machine learning model trained with second training data, a label image is output. The formats and combinations of input data and output data of a group of pairs constituting the training data are implemented so as to be suitable for an embodiment, such as a combination in which one is an image and the other is a numerical value, a combination in which one includes a plurality of groups of images and the other is a character string, or a combination in which both are images. In a case where training is performed using an image divided into rectangular regions, retinal layers are detected in each rectangular region. That is, a group of rectangular-region images with labels assigned for each pixel is obtained. The analysis unit 331 arranges the rectangular-region images with the labels assigned in the same relative positional relationship as in the original image and combines them, thereby obtaining a label image in which a retina is detected. Based on the output label image, boundaries between retinal layers are detected as boundary lines.

In the present embodiment, a boundary between an ILM and an NFL, a boundary between the NFL and a GCL, an ISOS, an RPE, and a BM are detected. Although not illustrated in the drawings, other boundaries may be detected, such as a boundary between an outer plexiform layer (OPL) and an outer nuclear layer (ONL), a boundary between the inner plexiform layer (IPL) and an inner nuclear layer (INL), a boundary between the INL and the OPL, and a boundary between the GCL and the IPL. Then, a process is performed to correct and smooth a shape of each detected boundary line. For example, coordinate values of each boundary line shape may be considered as time-series data of a signal, and the shape of each boundary line may be smoothed using a smoothing process such as a Savitzky-Golay filter, a simple moving average, a weighted moving average, or an exponential moving average.

### Step S305

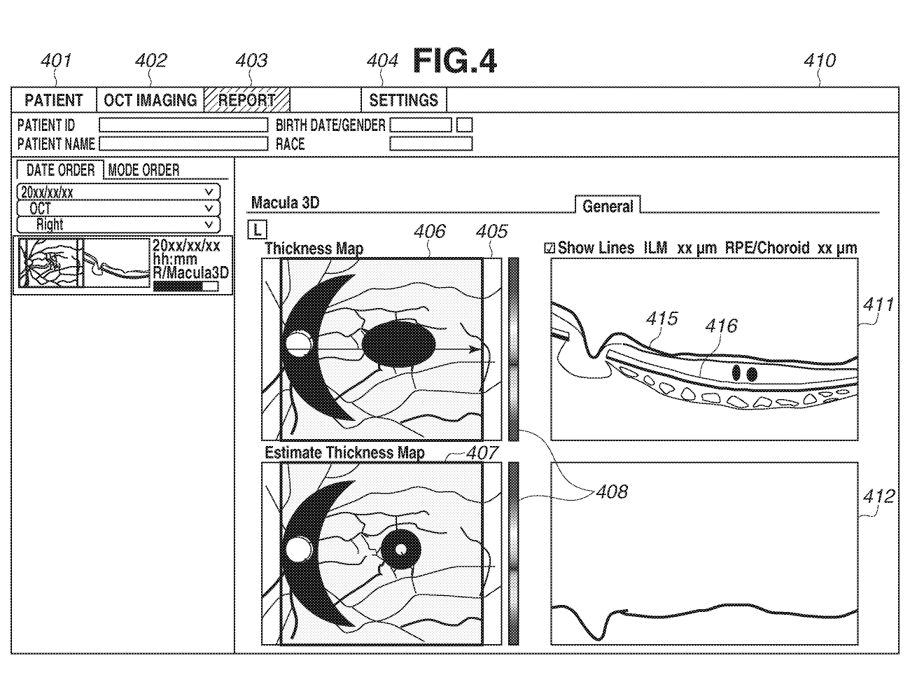

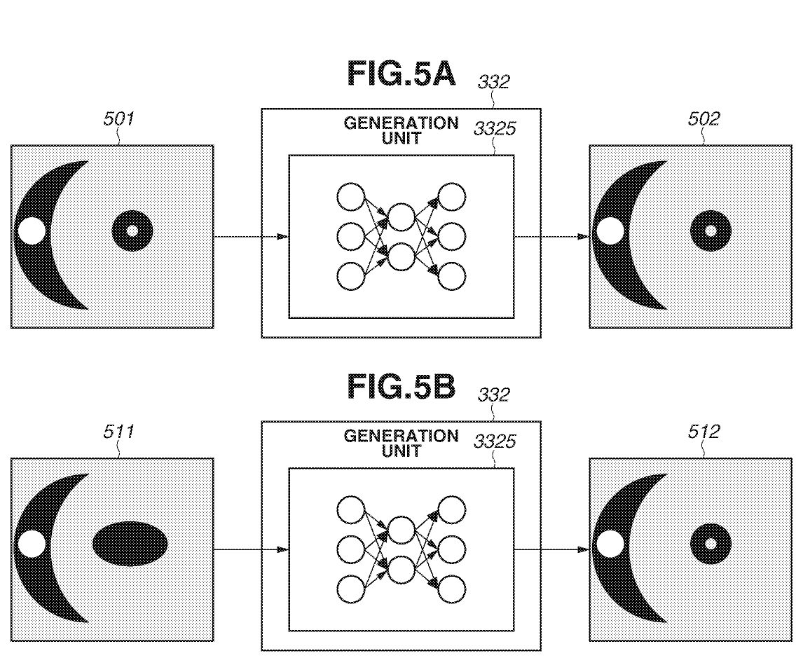

In step S305, the detected boundary lines and the tomographic image are displayed on the display unit 600. FIG. 4 illustrates an example of the screen displayed on the display unit 600. An entire screen 410 includes a patient tab 401, an imaging tab 402, a report tab 403, and a settings tab 404. Hatching in the report tab 403 indicates an active state of a report screen. In the present embodiment, an example of displaying a report screen (hereinafter referred to as a report screen 410) as the entire screen 410 will be described. The report screen 410 includes a tomographic image 411 in which boundary lines 415 and 416 are superimposed thereon as an example. The report screen 410 also includes a retinal thickness graph 412 obtained from the boundary lines 415 and 416. The report screen 410 also includes an SLO image 405 and a thickness map 406 that is a first analysis result obtained by analyzing the boundary lines obtained by the analysis unit 331. In the present embodiment, an example is illustrated in which thicknesses are obtained from the boundary lines 415 and 416 and in which a color thickness map, in which colors are assigned according to thickness values, is superimposed on the SLO image 405. A thickness map 407 is generated by the generation unit 332 as a second analysis result using, as an input, the thickness map 406. Similarly to the thickness map 406, the generated thickness map 407 is a color thickness map in which colors are assigned according to thickness values. Each color bar 408 indicates a relationship between retinal thickness and colors assigned according to thickness values when a thickness map is displayed on the display unit 600. The generation unit 332 is, for example, a model including an image processing system that outputs a thickness map by a rule-based processing or machine learning (particularly, deep learning technology). Hereinafter, a machine learning algorithm including an image processing system based on deep learning technology will be described with reference to FIGS. 5A, 5B, 6, 7A, and 7B. In the present embodiment, a machine learning model, for example, is a trained machine learning model 3325 that has been trained using training data including a group of thickness maps of normal eyes related to medical images.

The training data includes a group of thickness maps of one or more normal eyes. The machine learning model illustrated in FIGS. 5A and 5B is an example of an autoencoder. The autoencoder includes an encoder and a decoder. The encoder converts input data into low-dimensional data, and the decoder restores data from the low-dimensional data. The autoencoder is trained using a group of thickness maps corresponding to normal eyes. When inference is performed using the model 3325 trained with the group of thickness maps of normal eyes, in FIG. 5A, a thickness map 501 of a normal eye is converted into a tensor and input to the machine learning model 3325, and the generation unit 332 generates a still image based on the tensor output from the machine learning model 3325 and outputs a thickness map 502 resembling that of a normal eye. In FIG. 5B, a thickness map 511 of a diseased eye is converted into a tensor and input to the machine learning model 3325, and the generation unit 332 generates a still image based on the tensor output from the machine learning model 3325 and outputs a thickness map 512 resembling that of a normal eye. The thickness map 512 resembling that of a normal eye may be referred to as a thickness map estimated assuming that the diseased eye was normal. In the present embodiment, the tensor refers to a format in which pixel value groups of an image (a thickness map) and the like are represented as a multidimensional array, and is used as a data input/output format for the machine learning model 3325. The image (the thickness map) and the tensor are mutually convertible.

Examples of the thickness map include a thickness map of only the NFL, a thickness map of all layers from the ILM to the RPE, and a thickness map of a Ganglion Cell Complex (GCC) including the NFL, the GCL, and the IPL. In the present embodiment, a thickness map of all layers from the ILM to the RPE will be described as an example.

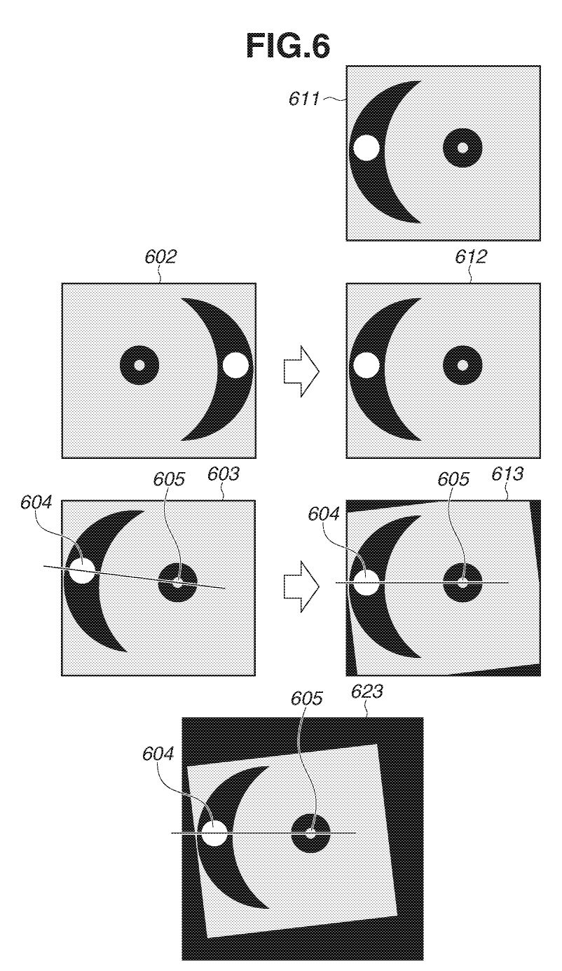

Next, an example of a case in which learning thickness maps are used for training will be described with reference to FIG. 6. FIG. 6 is a diagram illustrating preprocessing for training a model using thickness maps. FIG. 6 illustrates a full-layer thickness map 611 of a left eye, a full-layer thickness map 602 of a right eye, and a thickness map 612 that is an example obtained by horizontally inverting the thickness map 602. A thickness map 613 is an example of a full-layer thickness map obtained by rotating a thickness map 603 using position information of an optic nerve head 604 and a macular region 605 such that the positions of the optic nerve head 604 and the macular region 605 are aligned horizontally. The positions of the optic nerve head 604 and the macular region 605 are assumed to be detected by the image processing unit 303 when OCT data used as training data is captured. At the time of training, it is desirable that training be performed while the position information of characteristic portions is aligned to some extent, as illustrated in the thickness maps 611 to 613 in FIG. 6. In particular, for left and right eyes, horizontal inversion is performed to align the orientation to one of the left-eye orientation and the right-eye orientation. Although not illustrated, scaling of a thickness map may be performed such that the distance between the macular region and the optic nerve head becomes the same between different thickness maps. Furthermore, the thickness map may be shifted such that the macular region is located at a center position of the image. FIG. 6 illustrates the thickness maps obtained in a case where imaging is performed with the fixation lamp positioned at the center of the macula (or the center of the posterior pole). However, the present embodiment is not limited to this, and thickness maps obtained in a case where imaging is performed with the fixation lamp positioned at the center of the optic nerve head may be used. In this case, like in the example illustrated in FIG. 6, to align positional information of characteristic portions to some extent, the thickness map may be shifted such that the position information of the optic nerve head detected by the image processing unit 303 is aligned with the center of the image.

In the example of the thickness map 613, a rotated thickness map with the orientations of the optic nerve head 604 and macula 605 aligned horizontally is illustrated as having the same size as the thickness map before rotation, but the present embodiment is not limited to this. For example, as illustrated in a thickness map 623, in a case where a thickness map is rotated or shifted, an area that can accommodate the thickness map may be prepared, and the thickness map may be rotated within the area. For example, in a case where the size of the thickness map is 256 × 256, the size of the area is 300 × 300. In the example of the thickness map 623 in FIG. 6, the thickness map is rotated such that the positions of the optic nerve head 604 and the macular region 605 are aligned horizontally, and the macular region 605 is shifted to the center of the area. Expanding the area as illustrated in the thickness map 623 enables training to be performed without losing a part of the data even in a case where the thickness map is rotated or shifted. In the case where training is performed with an expanded area of a thickness map as illustrated above, the thickness maps 611 and 612 are also preprocessed to expand their sizes in the same way as the thickness map 623, and training is performed using the preprocessed thickness maps.

To evaluate accuracy and calculate an error (loss) between a thickness map in training data assigned for training and validation and a thickness map output from the machine learning model 3325, a calculation method based on the following technique may be employed. Specifically, for example, a method such as Mean Squared Error (MSE) and Structural Similarity (SSIM) may be used to quantify an error and similarity. In addition, in evaluating accuracy and calculating an error (loss) in training the machine learning model 3325, a target for the calculation may be selected taking into account a semantically meaningful area that is an area in a thickness map included in the training data and that can be segmented according to a pattern depicted in the thickness map or information related to the thickness map. Specifically, the semantically meaningful area indicates an area in which the thickness values of the retina are depicted. Areas that do not have such meaning include a blank margin area that appears when an image is rotated, as in the thickness map 613, and an expanded area, as in the thickness map 623. Since thickness values are not depicted in the blank marginal area or the expanded area (i.e., these areas do not affect diagnosis), in training the machine learning model 3325, only a semantically meaningful area that affects diagnosis may be set as a target for the accuracy evaluation and the error (loss) calculation, and the performance and characteristics of the machine learning model 3325 may be adjusted accordingly.

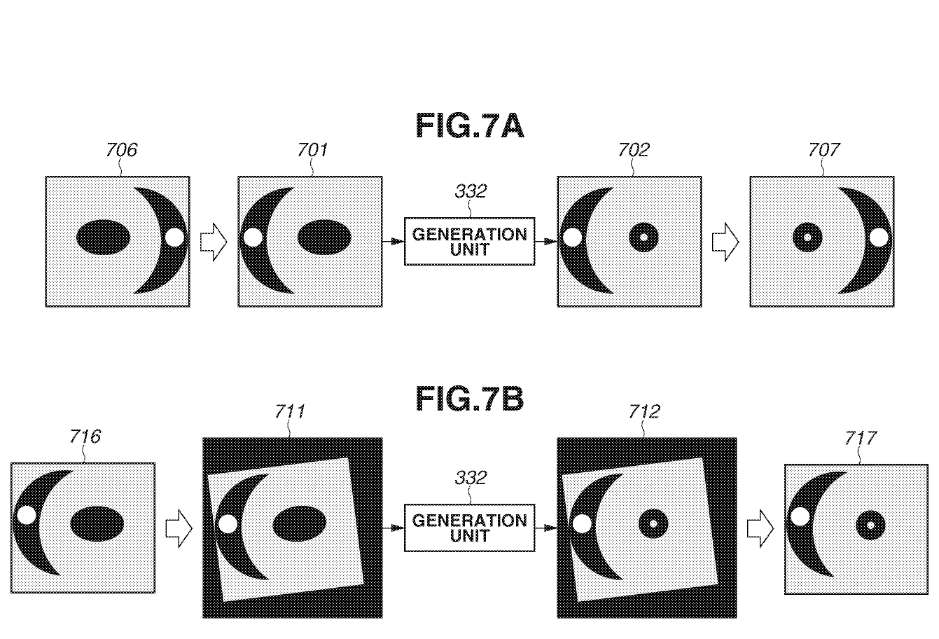

Next, an example of a case where a thickness map is inferred will be described with reference to FIGS. 7A and 7B. When the generation unit 332 generates a thickness map using the machine learning model 3325 that has been trained using a group of thickness maps as illustrated in FIG. 6, inference is performed after applying, to a thickness map, preprocessing similar to that during the training. FIG. 7A illustrates a case in which the machine learning model 3325 has been trained to match a left eye. In FIG. 7A, when a thickness map 706 of a right eye is input to the generation unit 332, the thickness map 706 of the right eye is horizontally inverted and input as a thickness map 701 to the generation unit 332, and an output thickness map 702 is horizontally inverted and displayed as a thickness map 707. In a case where the machine learning model 3325 has been trained to match a right eye, a thickness map of a left eye is horizontally inverted and input as a thickness map 701 to the generation unit 332.

Similarly, FIG. 7B illustrates an example of a case in which the machine learning model 3325 has been trained using an expanded area while adjusting an inclination using the position information of the optic nerve head and the macular region, as illustrated in the thickness map 623. In FIG. 7B, in the case of inputting a thickness map 716 to the generation unit 332, a thickness map 711 is generated by rotating the thickness map 716 within the area having the same size as used in training such that the positions of the optic nerve head and macular region are aligned horizontally. Then, the thickness map 711 is input to the generation unit 332, a thickness map 712 output from the generation unit 332 is rotated such that the positions of the optic nerve head and macular region are restored to their original positions, and the expanded area is clipped, so that a thickness map 717 that has been returned to the original size is displayed.

In the present embodiment, an example has been described in which, when training or inference is performed using a thickness map, the training and inference are performed using thickness values themselves, and, when the thickness map is displayed on the display unit 600, colors are assigned according to the thickness values. However, the present embodiment is not limited to this example. Training and inference may be performed using a color thickness-value map obtained by converting thickness values into colors. In this case, when the thickness map is displayed on the display unit 600, the inferred thickness map may be displayed without assigning color values according to thickness values.

### Step S306

In step S306, an instruction acquisition unit (not illustrated) acquires, from outside, an instruction indicating whether to end the tomographic imaging by the image processing system 100. This instruction is input by the operator using the input unit 700. Upon acquisition of an instruction to end the process, the image processing system 100 ends the process. On the other hand, in a case where imaging is to be continued without ending the process, the process returns to step S302 and imaging is continued. In the way described above, the processing of the image processing system 100 is performed.

According to the configuration described above, in the present embodiment, it is possible to infer a thickness map resembling that of a normal eye from an input thickness map. Since the inferred thickness map corresponds to the input, it is possible to display a thickness map in which only the thickness is represented as being normal while the structure of the eye to be examined is preserved.

### Modification 1 of first embodiment

In the present embodiment, an autoencoder has been described as an example of the machine learning model, but the present disclosure is not limited to this. For example, a variational autoencoder or a convolutional autoencoder may be used. Furthermore, a generative adversarial network (GAN) or a derivative thereof, such as AnoGAN, Efficient-GAN, and GANomary, may be used.

### Second Embodiment

In the first embodiment, an example has been described in which a thickness map inferred from a thickness map of an input image is displayed. In the present embodiment, difference information between the thickness map of the input image and the inferred thickness map is generated. Furthermore, difference information between the thickness map of the input image and statistical values of retinal thicknesses of normal eyes (normal-eye database) is generated and displayed to the operator.

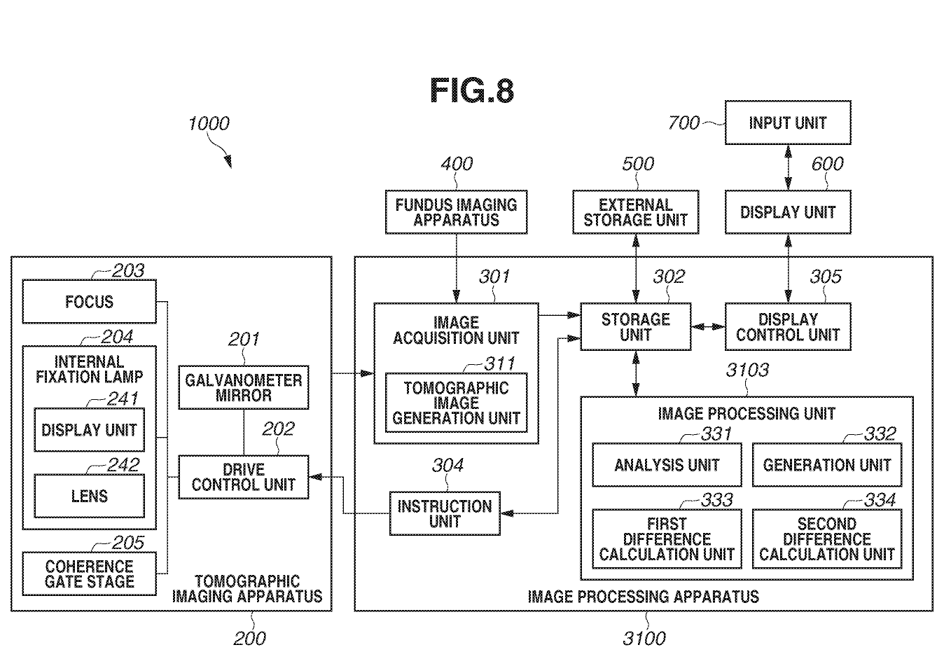

FIG. 8 is a diagram illustrating an example of a schematic configuration of an image processing system 1000 including an image processing apparatus 3100 according to the second embodiment. In FIG. 8, the same reference numerals are assigned to components similar to those in the configuration illustrated in FIG. 1, and detailed descriptions thereof will be omitted. Here, an image processing unit 3103, which is different from that of the first embodiment, in the image processing system 1000 illustrated in FIG. 8 will be described. The image processing unit 3103 includes a first difference calculation unit 333 and a second difference calculation unit 334. The first difference calculation unit 333 calculates difference information (hereinafter, also referred to as a difference thickness map) between a retinal-layer thickness map that is a first analysis result, and a thickness map generated by the generation unit 332 that is a second analysis result. The second difference calculation unit 334 calculates difference information between the retinal layer thickness map that is the first analysis result and statistical values of retinal-layer thicknesses of a plurality of eyes stored in the storage unit 302. The display unit 600 displays at least two of the retinal-layer thickness map that is the first analysis result, the retinal-layer thickness map that is the second analysis result, the difference information generated by the first difference calculation unit 333, and the difference information generated by the second difference calculation unit 334.

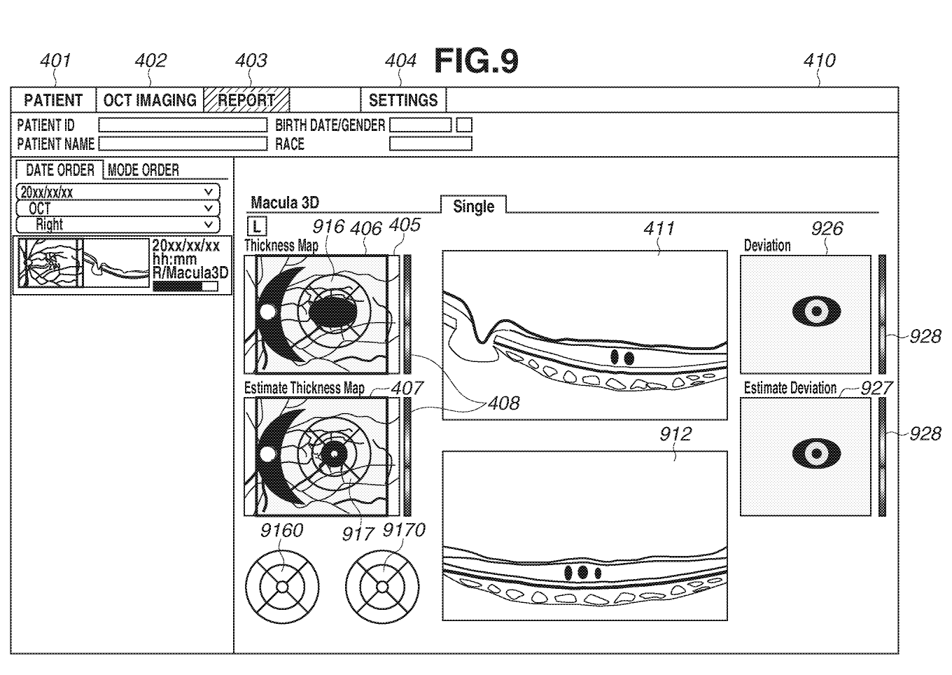

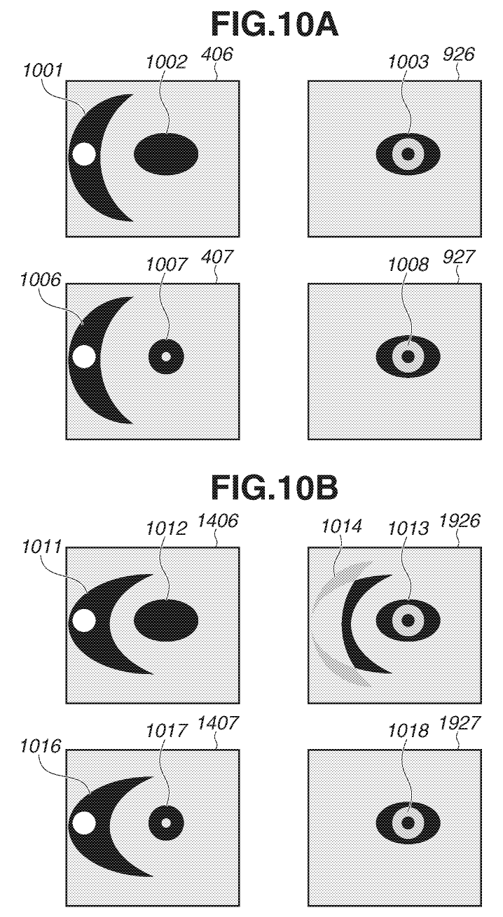

This will be described with reference to FIG. 9. FIG. 9 illustrates a report screen 410 displayed on the display unit 600 in the present embodiment, and is an example in which the screen is displayed in a different layout from that illustrated in FIG. 4. FIG. 9 illustrates an example of displaying a retinal-layer thickness map 406, a thickness map 407 generated by the generation unit 332, a difference thickness map 927 generated by the first difference calculation unit 333, and a difference thickness map 926 generated by the second difference calculation unit 334, together with a tomographic image 411 and a tomographic image 912 in a vertical cross-sectional plane. The thickness maps and the difference thickness maps will be described with reference to FIGS. 10A and 10B. In FIG. 10A, the thickness map 406 is the first analysis result generated from retinal layers analyzed by an analysis unit, and illustrates an example in which the course of nerve fibers 1001 is typical and the thickness is normal, while swelling is observed around a macular region 1002 due to a disease. The thickness map 407 is a thickness map that is generated by the generation unit 332 using the thickness map 406 as an input, and illustrates an example in which the thickness around the macular region is output to be close to that of a normal eye. The difference thickness map 927 is difference information generated by the first difference calculation unit 333, and is a difference thickness map between the thickness maps 406 and 407. The difference thickness map 926 is difference information generated by the second difference calculation unit 334, and is a difference thickness map between the thickness map 406 and the statistical values of retinal-layer thicknesses of a plurality of eyes stored in the storage unit 302. The differences calculated by the first difference calculation unit 333 and the second difference calculation unit 334 each represent a difference between different thickness maps, such as a difference or a ratio in a plurality of thickness maps. As illustrated in FIG. 10A, in the case of a typical eye, difference portions 1003 and 1008 based on swelling information around the macular region are respectively displayed as the difference information.

In FIG. 10B, a thickness map 1406 is the first analysis result generated from retinal layers analyzed by the analysis unit, and illustrates an example in which the course of nerve fibers 1011 is not typical but the thickness is normal, while swelling is observed around a macular region 1012 due to a disease. A thickness map 1407 is a thickness map generated by the generation unit 332 using the thickness map 1406 as an input, and illustrates an example in which the thickness around the macular region is output to be close to that of a normal eye. A difference thickness map 1927 is difference information generated by the first difference calculation unit 333, and is a difference thickness map between the thickness maps 1406 and 1407. A difference thickness map 1926 is difference information generated by the second difference calculation unit 334, and is a difference thickness map between the thickness map 1406 and the statistical values of retinal-layer thicknesses of a plurality of eyes stored in the storage unit 302. As illustrated in the difference thickness map 1926, difference information 1014 is displayed in an area that deviates from the statistical values even though the thickness is within the normal range. As described above, since the generation unit 332 generates a thickness map based on the input thickness map, the generation unit 332 generates a thickness map based on learned information so that the generated thickness map is less affected by individual differences in shape, thickness, and axial length.

In the present embodiment, thickness maps and difference information as illustrated in FIGS. 10A and 10B are displayed on the report screen 410. Furthermore, on the report screen 410, grids 916 and 917 are superimposed on the retinal-layer thickness maps 406 and 407, respectively, and numerical values in the respective grids are displayed in grids 9160 and 9170. Although not illustrated, the numerical values in the grids may be displayed not only in the grids but also in a tabular form or a graph form.

In the present embodiment, as an example has been described in which the thickness maps and the difference thickness maps described above are displayed side by side on the report screen 410, but the present embodiment is not limited to this. For example, one of the thickness map generated by the analysis unit 331 and the thickness map generated by the generation unit 332 may be displayed in a switchable manner, and accordingly, the difference thickness map may be switched and displayed.

According to the configuration described above, in the present embodiment, the operator can make an integrated determination by referring to one or more of the thickness map obtained by the analysis unit through analysis of retinal layers, the thickness map generated by the generation unit, and the difference information generated by the first difference calculation unit and the second difference calculation unit.

### Third Embodiment

In the first and second embodiments, examples have been described in which thickness maps are generated and difference information between the thickness maps is generated. In the present embodiment, an example will be described in which a condition acquisition unit is provided to acquire imaging conditions and thickness map conditions, and thickness maps are generated based on these conditions.

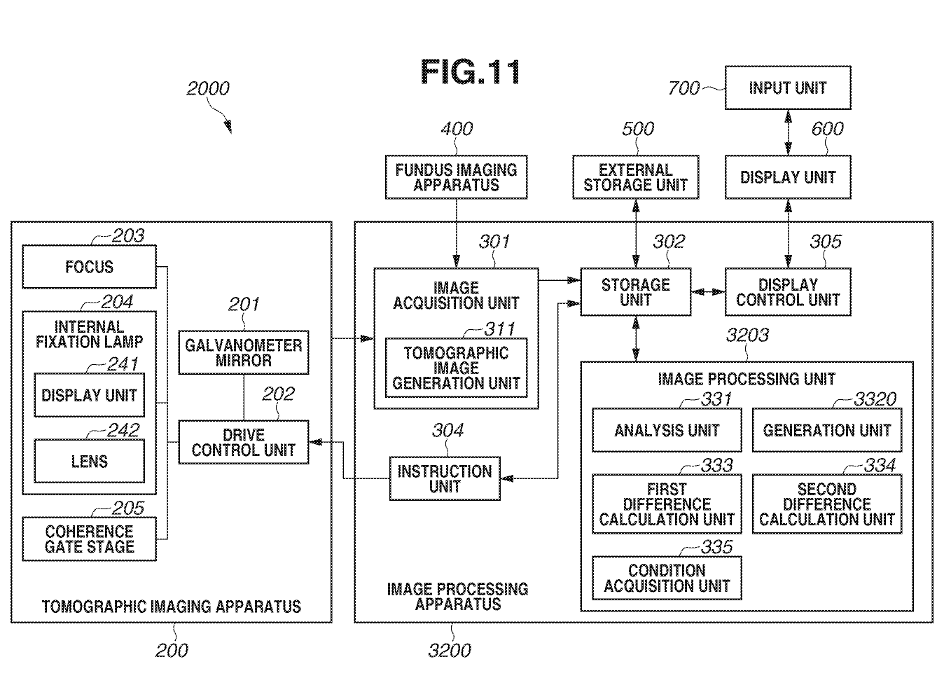

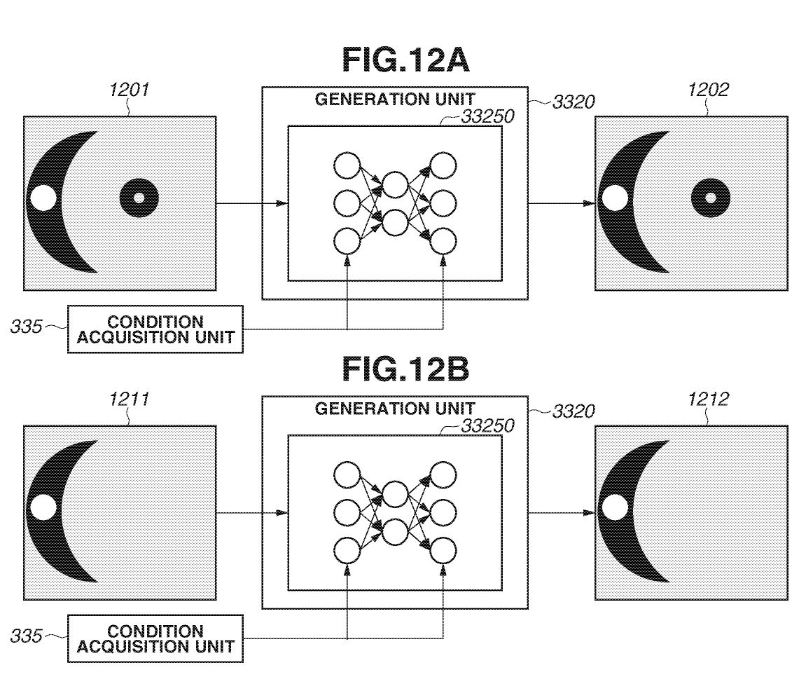

FIG. 11 is a diagram illustrating an example of a schematic configuration of an image processing system 2000 including an image processing apparatus 3200 according to the third embodiment. In FIG. 11, the same reference numerals are assigned to components similar to those illustrated in FIGS. 1 and 8, and detailed descriptions thereof will be omitted. Here, an image processing unit 3203, which is different from those of the first and second embodiments, in the image processing system 2000 illustrated in FIG. 11 will be described. The image processing unit 3203 includes a condition acquisition unit 335. A generation unit 3320 generates a thickness map based on conditions acquired by the condition acquisition unit 335. In this regard, a machine learning algorithm including an image processing system based on deep learning technology will be described with reference to FIGS. 12A and 12B. FIGS. 12A and 12B are diagrams illustrating a machine learning model 33250 included in the generation unit 3320 according to the third embodiment.

Specifically, the machine learning model 33250 illustrated in FIG. 12A receives, as inputs, a thickness map 1201 converted into a tensor and a thickness-map tensor to which scalar values indicating the conditions are added. The generation unit 3320 then converts the tensor output from the machine learning model 33250 into a still image and outputs the still image as a thickness map 1202.

Examples of conditions acquired by the condition acquisition unit 335 include imaging conditions, conditions of an eye to be examined, and analysis conditions. Examples of the imaging conditions include information regarding the date and time of imaging, the name of a site imaged, an imaging region, an angle of view, an imaging method, a resolution and gradation of an image, a pixel size of the image, an image filter, and a data format of the image. Examples of the information regarding the eye to be examined include an age (date of birth), sex, and race. The analysis conditions are, for example, information regarding retinal layers included in a thickness map. The information regarding retinal layers refers to information regarding layers included in a thickness map generated from a tomographic image (i.e., a type of thickness map). Examples of the type of the thickness map include a thickness map having a thickness of each of the layers, a thickness map having a thickness of all layers from an ILM to a RPE, and a GCC thickness map including an NFL, a GCL, and an IPL.

FIG. 12A illustrates an example in which, together with the thickness map 1201, a condition indicating that the thickness map is a full-layer thickness map is input as an analysis condition, and a thickness map corresponding to the condition is output. FIG. 12B illustrates an example in which, together with a thickness map 1211, a condition indicating that the thickness map is a GCC thickness map is input as an analysis condition, and a thickness map 1212 corresponding to the condition is output. For example, a scalar value of the analysis condition may be set to 001001 for a full-layer thickness map, and to 001002 for a GCC thickness map. In addition to the conditions in the cases of FIGS. 12A and 12B, other conditions may also be input, such as the center of the macula as an imaging site, 10 × 10 mm as an imaging region, and information on the eye to be examined, such as age, sex, race, and measurement values regarding an eye (e.g., an axial length and a refractive value) as information regarding an eye to be examined. The condition added to a thickness map need not be a single condition, and a plurality of conditions may be added simultaneously. Alternatively, a plurality of conditions may be represented by a single scalar value.

According to the configuration described above, in the present embodiment, by learning conditions together with thickness maps, it is possible to simultaneously learn various types of thickness maps. At the time of inference, by inputting conditions together with a thickness map, it is possible to output a thickness map based on the input conditions.

### Fourth Embodiment

In the present embodiment, an example will be described in which time-series thickness maps are displayed. The time-series thickness maps are thickness maps generated using a plurality of tomographic images captured at different times. Descriptions of components having functions similar to those in the first to third embodiments described above will be omitted here.

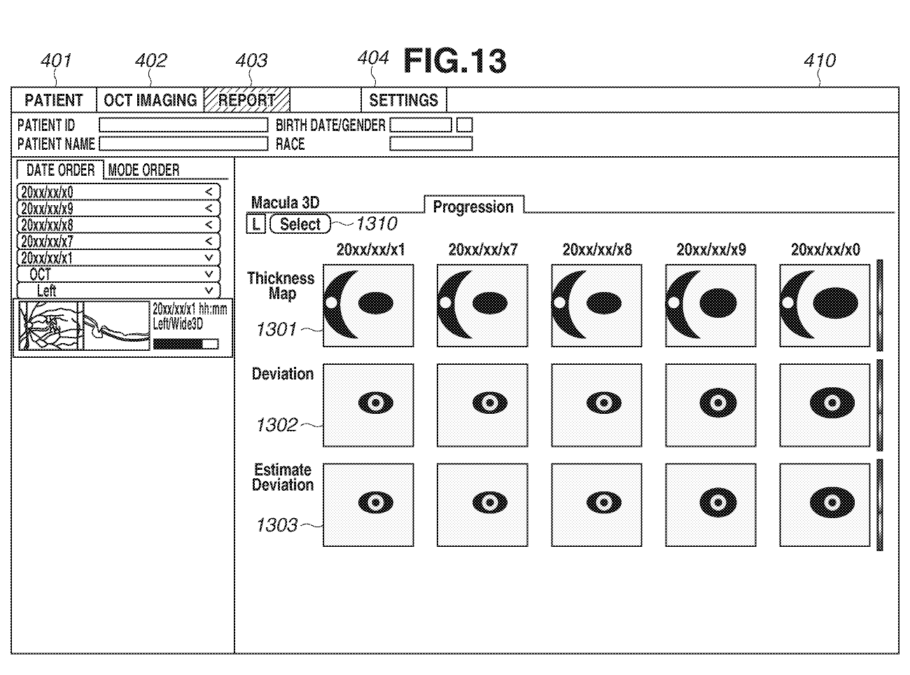

FIG. 13 illustrates an example of displaying time-series thickness maps in the present embodiment. In FIG. 13, the time-series thickness maps are displayed side by side in a horizontal direction. Difference thickness maps are displayed below the respective thickness maps and arranged in a vertical direction. A thickness map (Thickness Map) 1301 is generated by an analysis unit. A difference thickness map 1302 (Deviation) is a difference thickness map between the retinal-layer thickness map 1301 and statistical values of retinal-layer thicknesses stored in the storage unit 302, and the difference thickness map 1302 is generated by the second difference calculation unit 334. A difference thickness map (Estimate Deviation) 1303 is a difference thickness map between a retinal-layer thickness map that is a first analysis result and a thickness map that is a second analysis result generated by the generation unit 332, and the difference thickness map 1303 is generated by the first difference calculation unit 333.

As illustrated in the present embodiment, a plurality of thickness maps and a plurality of difference thickness maps can be displayed. For each difference thickness map 1303 generated by the first difference calculation unit 333, a thickness map (second analysis result) inferred from a thickness map (first analysis result) obtained at a certain reference time may be used as a reference map, and a difference from the reference map may be displayed. Alternatively, a difference between a thickness map (first analysis result) obtained at each time and a thickness map (second analysis result) inferred therefrom may be displayed for each time. Alternatively, the image processing unit 3203 may include an integration unit (not illustrated), and the integration unit may generate a statistical thickness map (for example, an average map obtained by averaging the respective second analysis results) by using a plurality of thickness maps (second analysis results) inferred respectively from thickness maps (first analysis results) obtained at the respective times, and a difference from the statistical thickness map may be displayed. The statistical thickness map obtained by integrating the respective second analysis results is an example of a third analysis result.

Further, although not illustrated, the thickness maps generated by the generation unit 332 at the respective times may be displayed. These thickness maps may be displayed side by side, or may be displayed in a switchable manner. Furthermore, the thickness maps are not limited to time-series data, and a thickness map may be generated from each of the thickness maps of the left and right eyes, and then a difference thickness map may be generated.

According to the configuration described above, in the present embodiment, it is possible to infer a thickness map close to that of a normal eye from a plurality of input thickness maps, such as time-series thickness maps or left-eye and right-eye thickness maps. Furthermore, it is possible to simultaneously display a difference from the statistical values of normal-eye thicknesses and a difference from an inferred thickness map.

### Modification 1

In the machine learning models in the various embodiments described above, an example has been described in which training is performed using only one type of thickness map, and an example in which training is performed using a plurality of types of thickness maps and conditions (e.g., imaging conditions, conditions of an eye to be examined, analysis conditions, and the like) to support a plurality of types. However, the configuration is not limited to these examples. For example, a configuration may be employed in which a machine learning model trained using one type or several types of thickness maps for each condition is prepared, and a machine learning model to be used may be switched according to a condition. As an example of switching machine learning models trained for respective conditions, for each type of thickness map such as a single-layer thickness map, a full-layer thickness map, and a GCC thickness map, a machine learning model may be trained together with conditions of an eye to be examined such as race, age, and sex. Then, at the time of inference, a machine learning model may be selected according to an analysis condition (a type of a thickness map to be used as an input), and inference may be performed using the thickness map together with the conditions of the eye to be examined. Not limited to this example, machine learning models may be trained using a plurality of types of thickness maps collectively for each condition of an eye to be examined (e.g., race, sex, age, and the like), and a machine learning model to be used at the time of inference may be selected according to the condition of the eye to be examined. Regarding age, instead of dividing the age on one-year basis, ages may be grouped with a certain range, such as the 30s to 40s or the 50s to 60s.

### Modification 2

In the machine learning models of the various embodiments described above, an example has been described in which training is performed using training data including a group of thickness maps of normal eyes, but the configuration is not limited to this. For example, training may be performed using training data including a group of thickness maps of diseased eyes. In this case, in the machine learning model, when a thickness map of a normal eye is input, a thickness map resembling that of a diseased eye is output. When a thickness map of a diseased eye is input, a thickness map of a diseased eye is output. Thus, the closer the eye is to a normal eye, the greater the difference becomes when difference information between the input thickness map and the output thickness map is generated. A machine learning model may be trained for each type of disease. Examples of the types of diseases may include glaucoma, diabetic retinopathy, age-related macular degeneration, and the like. Accordingly, by performing inference using machine learning models trained for the respective diseases, it is possible to predict a disease, for example, in such a manner a disease that results in the smallest difference between the input thickness map and the output thickness map is most likely to be the disease.

### Other embodiments

The present disclosure may also be realized by executing the following processing. That is, the present disclosure may be realized by supplying software (a program) for implementing one or more functions of the various embodiments described above to a system or apparatus via a network or a storage medium, and causing a computer (or a CPU, an MPU, or the like) of the system or apparatus to read out and execute the program. The computer may include one or more processors or circuits, and may include a network of separate computers or a network of separate processors or circuits for reading out and executing computer-executable instructions. In this case, the processors or circuits may include a central processing unit (CPU), a micro processing unit (MPU), a graphics processing unit (GPU), an application specific integrated circuit (ASIC), or a field programmable gate array (FPGA). Further, the processors or circuits may include a digital signal processor (DSP), a data flow processor (DFP), or a neural processing unit (NPU).

According to an embodiment of the present disclosure, it is possible to improve the accuracy of diagnosis.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

The present disclosure is not limited to the embodiments described above, and various changes and modifications are possible without departing from the spirit and scope of the present disclosure. Accordingly, the following claims are appended hereto to make public the scope of the present disclosure.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. An information processing apparatus comprising:
a generation unit configured to generate, based on a tomographic image of an eye to be examined, a first analysis result having two-dimensional thickness data of a retinal layer of the eye to be examined, and generate a second analysis result different from the first analysis result using a machine learning model trained with a plurality of thickness maps corresponding to a plurality of normal eyes and the first analysis result; and
a display control unit configured to control a display unit to display information regarding the second analysis result.

2. The information processing apparatus according to claim 1, wherein the display control unit controls the display unit to display, as the information regarding the second analysis result, a comparison result between the first analysis result and the second analysis result.

3. The information processing apparatus according to claim 1, wherein the display control unit controls the display unit to display at least one of a comparison result between the first analysis result and the second analysis result, and a comparison result between the first analysis result and a thickness map acquired from a normal-eye database.

4. The information processing apparatus according to claim 1, wherein the generation unit generates the second analysis result different from the first analysis result, using the machine learning model, the first analysis result, and information regarding the eye to be examined.

5. The information processing apparatus according to claim 4, wherein the information regarding the eye to be examined is at least one of a race of a subject, an age of the subject, a sex of the subject, and an axial length of the eye to be examined.

6. The information processing apparatus according to claim 1, wherein the generation unit generates the second analysis result different from the first analysis result, using the machine learning model, the first analysis result, and information regarding the retinal layer included in the first analysis result.

7. The information processing apparatus according to claim 6, wherein the information regarding the retinal layer is layer information included in the first analysis result.

8. The information processing apparatus according to claim 1, wherein the display control unit controls the display unit to display the first analysis result and the second analysis result side by side.

9. The information processing apparatus according to claim 1, wherein the display control unit controls the display unit to display side by side a comparison result between the first analysis result and the second analysis result, a comparison result between the first analysis result and a thickness map acquired from a normal-eye database, the first analysis result, and the second analysis result.

10. The information processing apparatus according to claim 1, wherein the display control unit controls the display unit to display side by side:
a plurality of first analysis results each generated using a corresponding one of a plurality of tomographic images obtained by imaging the eye to be examined at different times;
a plurality of comparison results each being a comparison result between a corresponding one of the plurality of first analysis results and a thickness map acquired from a normal-eye database; and
a plurality of comparison results each being a comparison result between a corresponding one of the plurality of first analysis results and a second analysis result generated using the machine learning model and at least one of the plurality of first analysis results.

11. The information processing apparatus according to claim 1, wherein the display control unit controls the display unit to display side by side:
a plurality of first analysis results each generated using a corresponding one of a plurality of tomographic images obtained by imaging the eye to be examined at different times;
a plurality of comparison results each being a comparison result between a corresponding one of the plurality of first analysis results and a thickness map acquired from a normal-eye database; and
a plurality of comparison results each being a comparison result between a corresponding one of the plurality of first analysis results and a third analysis result obtained by averaging a plurality of second analysis results generated using the machine learning model and the plurality of first analysis results.

12. The information processing apparatus according to claim 1,
wherein the generation unit includes the machine learning model configured to receive, as an input, the first analysis result of one of a right eye and a left eye of the eye to be examined,
wherein, in a case where the first analysis result of the one eye is input, the generation unit generates the second analysis result by inputting the first analysis result of the one eye into the machine learning model, and
wherein, in a case where the first analysis result of the other of the right eye and the left eye of the eye to be examined is input, the generation unit generates the second analysis result by horizontally inverting the first analysis result of the other eye and inputting the horizontally inverted first analysis result into the machine learning model.

13. An information processing apparatus comprising:
an analysis unit configured to generate, based on a tomographic image of an eye to be examined, a first analysis result having two-dimensional thickness data of a retinal layer of the eye to be examined; and
a display control unit configured to control a display unit to display information regarding a second analysis result different from the first analysis result, the second analysis result being generated using a machine learning model trained with a plurality of thickness maps corresponding to a plurality of normal eyes and the first analysis result.

14. An information processing system comprising:
an optical coherence tomography (OCT) apparatus configured to capture a tomographic image of an eye to be examined; and
the information processing apparatus according to claim 1 communicably connected to the OCT apparatus.

15. An information processing method comprising:
generating, based on a tomographic image of an eye to be examined, a first analysis result having two-dimensional thickness data of a retinal layer of the eye to be examined;
generating a second analysis result different from the first analysis result using a machine learning model trained with a plurality of thickness maps corresponding to a plurality of normal eyes and the first analysis result; and
controlling a display unit to display information regarding the second analysis result.

16. An information processing method comprising:
generating, based on a tomographic image of an eye to be examined, a first analysis result having two-dimensional thickness data of a retinal layer of the eye to be examined; and
controlling a display unit to display information regarding a second analysis result different from the first analysis result, the second analysis result being generated using a machine learning model trained with a plurality of thickness maps corresponding to a plurality of normal eyes and the first analysis result.

17. A non-transitory computer-readable storage medium storing a program for causing a computer to perform the information processing method according to claim 15.

18. A non-transitory computer-readable storage medium storing a program for causing a computer to perform the information processing method according to claim 16.
